(19) United States
(12) Patent Application Publication
(10) Pub. No.: US 2025/0144080 A1
(43) Pub. Date: May 8, 2025

(54) COMPOSITIONS AND METHODS FOR THE TREATMENT OF MACULAR DEGENERATION

(71) Applicant: Aldeyra Therapeutics, Inc., Lexington, MA (US)

(72) Inventors: Thomas A. Jordan, Lexington, MA (US); John Clifford Chabala, Scotch Plains, NJ (US); Gerald D. Cagle, Fort Worth, TX (US)

(21) Appl. No.: 18/786,975

(22) Filed: Jul. 29, 2024

Related U.S. Application Data
(63) Continuation of application No. 18/241,598, filed on Sep. 1, 2023, now Pat. No. 12,097,188.
Application No. 18/241,598 is a continuation of application No. 15/782,470, filed on Oct. 12, 2017, now abandoned.
Application No. 15/782,470 is a continuation of application No. 13/514,769, filed on Aug. 17, 2012, now Pat. No. 9,814,701.
(60) Provisional application No. 61/285,745, filed on Dec. 11, 2009.

(51) Int. Cl. A61K 31/423; A61K 9/00; A61K 31/47; A61K 47/40

(52) U.S. Cl. CPC A61K 31/423; A61K 9/0048; A61K 31/47; A61K 47/40

(57) ABSTRACT

The present invention is directed to topical ophthalmic compositions of a lipophilic compound and an oligomeric or polymeric carrier wherein the compositions are useful in the treatment and prevention of macular degeneration. The invention also includes methods of treating macular degeneration by using a topical ophthalmic composition of a lipophilic compound and an oligomeric or polymeric carrier.

### RELATED APPLICATION

This application claims the benefit of U.S. Provisional Application No. 61/285,745, filed Dec. 11, 2009, the entire contents of which is incorporated herein by reference.

### BACKGROUND OF THE INVENTION

Macular degeneration is a leading cause of progressive blindness. The macula is the central region of the retina and contains the fovea where high acuity central vision is processed. Macular degeneration is a neurodegenerative disease in the macula that progressively causes disabling deficits in visual function.

There are multiple forms of macular degeneration. Age-related macular degeneration (AMD) is the most common form and first appears at middle age or later. In AMD patients, the dry form normally occurs first and develops with no vascular complications. Its clinical signs include an increase in fundus auto-fluorescence (FAF) and the formation of extracellular deposits called soft drusen, both caused by the accumulation of lipofuscin in retinal pigment epithelial (RPE) cells as discussed below. About 40% of dry AMD patients progress to an advanced form of the disease called geographic atrophy (GA) secondary to dry AMD, which is characterized by one or more atrophic retinal lesions caused by the localized death of RPE cells and adjacent retinal photoreceptor cells. Another 10% of dry AMD patients progress to wet AMD, which is characterized by neovascular growth from the choroid into the retina in response to VEGF signaling by RPE cells that are undergoing severe oxidative stress from A2E toxicities. Choroidal neovascular growth disrupts retinal tissue and destroys visual function in regions of the macula where this occurs. Finally, there is an early onset form of macular degeneration called Stargardt's disease which first appears in teenagers and young adults. Stargardt's disease is believed to have the same etiology as dry AMD.

Multiple lines of evidence indicate that macular degeneration is caused by the cytotoxic accumulation in RPE cells of naturally occurring bis-retinoid compounds including A2E (Sparrow J R, Wu Y, Kim C Y, Zhou J. Phospholipid meets all-trans-retinal: the making of RPE bisretinoids. J Lipid Res. 2009 Aug. 7 e-published). A2E is a reaction product of all-trans retinaldehyde (RAL) and phosphatidylethanolamine (PE), a membrane phospholipid found in the disc membranes of photoreceptor outer segments. The RAL that reacts with PE escapes from the visual cycle (step 3b in FIG. 1), a metabolic pathway in the back of the eye that (i) converts vitamin A from an alcohol (retinol) to a photo-reactive aldehyde (11-cis-retinaldehyde) for use in photo-transduction by opsin proteins in photoreceptor cell outer segments, and (ii) converts RAL to retinol after photo-transduction. As RAL escapes the visual cycle, A2E precursors form reversibly in photoreceptor outer segments, which are ingested by neighboring RPE cells after diurnal shedding. The final and irreversible step in the biosynthesis of A2E takes place in the acidic environment of RPE cell lysosomes. As A2E accumulates in RPE cells, it gradually poisons them by multiple cytotoxic mechanisms including lysosomal failure. This leads to the accumulation of undigested cellular debris called lipofuscin, which contains A2E and can be detected clinically by FAF. As RPE cells deteriorate, they lose their ability to participate in the visual cycle and are unable to provide photoreceptors with other metabolic support required for normal retinal function (see FIG. 1; Lamb T D, Pugh E N, Dark adaptation and the retinoid cycle of vision. Prog. Retinal and Eye Res. 2004;23:307). As their metabolic support is withdrawn, photoreceptors fail to renew their shed outer segments and visual function is progressively lost. Reducing A2E formation will allow RPE cells to recover from A2E poisoning and resume their normal metabolic support of photoreceptor cells.

WO 2006/127945 discloses compounds and compositions that have been shown to reduce the formation of A2E, which as described above is the underlying etiology of macular degeneration including dry AMD and Stargardt's disease. These compounds are designed to inhibit A2E biosynthesis by reducing the amount of free RAL available for reaction with PE in photoreceptor outer segments, which is the first step in the A2E biosynthetic pathway. These compounds are lipophilic by design (i.e. the logarithm of their measured or calculated partition constant between water and n-octanol [logP or clogP which are called logD or clogD respectively at pH 7.4 as used herein] is greater than 0), because their covalent reactions with RAL take place in the disc membranes of photoreceptor outer segments where A2E precursors form.

A different way to reduce A2E biosynthesis is to inhibit one or more proteins of the visual cycle, because when the visual cycle is inhibited, less RAL escapes the visual cycle and becomes available to react with PE and form A2E precursors. Chronic treatment with a visual cycle inhibitor has been shown to reduce A2E synthesis in mouse (Radu et al., Treatment with isotretinoin inhibits lipofuscin accumulation in a mouse model of recessive Stargardt's macular degeneration. Proc Natl Acad Sci U S A. 2003;100:4742). The clinical value of this treatment approach is limited by the fact that visual cycle inhibitors cause night blindness by lowering rod photoreceptor sensitivity, impair dark adaptation by slowing the dark adaptation rate, and cause retinoid toxicities by activating retinoic acid receptors (RAR) if the visual cycle inhibitor compound is a member of the retinoid class. Visual cycle inhibitors now in clinical development include fenretinide which is a retinoid compound, and ACU-4429 which reportedly is not a retinoid.

Other approaches to treating macular degeneration are based on neuroprotection mechanisms of action including but not limited to neurotrophic receptor agonists, anti-inflammatory compounds including complement cascade inhibitors, anti-apoptosis compounds, steroids and anti-oxidant compounds, and limiting the progression to wet AMD with VEGF receptor blockers which mitigate the effects of VEGF signaling by RPE cells that are in severe oxidative stress as a consequence of A2E toxicities.

### SUMMARY OF THE INVENTION

In therapeutic and prophylactic use, topical ocular administration minimizes systemic exposure compared to oral dosing. The present invention provides formulations and methods for topical ocular dosing of lipophilic compounds which treat macular degeneration (including dry AMD, GA secondary to dry AMD, wet AMD and Stargardt's disease) by reducing the formation of A2E and other naturally occurring bis-retinoids, by reacting covalently with free RAL that escapes the visual cycle and would otherwise form precursors of these compounds. The lipophilic compounds within the scope of this invention are further defined by having a logP or clogP (logD or clogD at pH=7.4) greater than 0.

### BRIEF DESCRIPTION OF THE FIGURES

FIG. 1 depicts the visual cycle.

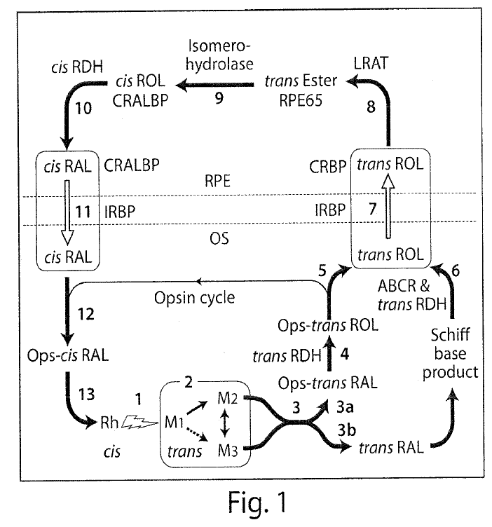

FIG. 2 depicts a time profile of the concentration of ¹⁴C-Compound A in ng/g of ocular tissue.

FIG. 3 depicts effects of RA treatment on dark adaptation rate in the mouse after acute TO or IP administration of 13-cis-retinoic acid.

### DETAILED DESCRIPTION OF THE INVENTION

One embodiment of the present invention provides pharmaceutical compositions for treating macular degeneration (including dry AMD, GA secondary to dry AMD, wet AMD and Stargardt's disease). The root cause of macular degeneration is the cytotoxic accumulation in RPE cells of naturally occurring bis-retinoids including A2E. This accumulation can be lowered pharmacologically by limiting the amount of free RAL that escapes the visual cycle and otherwise reacts with PE to form their precursors. As the accumulation of these compounds is reduced pharmacologically, RPE cells can recover from cytotoxic damage and resume their metabolic support of photoreceptors which is essential for normal visual function. In publication WO 2006/127945, compounds are described which reduce A2E accumulation in this manner and are therefore useful in treating macular degeneration. The instant application incorporates by reference the subject matter of WO 2006/127945.

Lipophilic compounds which reduce A2E and thereby treat macular degeneration including dry AMD and Stargardt's disease by other means (e.g. by inhibiting the visual cycle) are also included within the scope of this invention. Such compounds include ACU-4429. All such compounds have a logP or clogP greater than 0.

In therapeutic or prophylactic use, topical ocular administration minimizes systemic exposure compared to oral dosing. However, most topical formulations have failed to deliver efficacious levels of drug to the retina which is the site of the mechanism of action. Surprisingly, applicants have found that by employing preparations containing a lipophilic active agent and a cyclodextrin, or chemically modified cyclodextrin including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt), efficacious levels of the lipophilic active agent are delivered to the back of the eye and specifically to the RPE and retina. Pharmaceutical compositions of a lipophilic active agent and an oligomeric or a polymeric carrier such as a cyclodextrin or chemically modified cyclodextrin including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt) are useful for the treatment of macular degeneration (including dry age-related macular degeneration, GA secondary to dry AMD, wet AMD and Stargardt's disease).

One exemplification of the present invention is represented by a topical ophthalmic composition containing an active lipophilic compound and an oligomeric or a polymeric carrier such as a cyclodextrin, or a chemically modified cyclodextrin including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt) in an aqueous solution or a gel dispersion. Such active lipophilic compounds include those that reduce A2E by reacting covalently with RAL, e.g. the compounds of WO 2006/127945. Illustrative of such compounds are those described in WO 2006/127945 the contents of which application are herein incorporated by reference. Exemplifying the compounds which may be employed in the instant invention are the compounds of formula Ia:

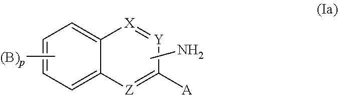

and pharmaceutically acceptable salts thereof, wherein X, Y, and Z are each, independently, N, CH, C with the NH₂ attached, or absent, such that one of X, Y, and Z is N; p is 0, 1, 2, or 3, B is a halogen atom, hydroxyl, carbamoyl, amino, or aryl, A is

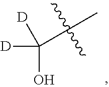 [SMILES: *C([2H])([2H])O]

D is unbranched lower alkyl. Specifically, compound A of paragraph (00027) of WO 2006/127945 and pharmaceutically acceptable salts thereof may be employed in the instant invention.

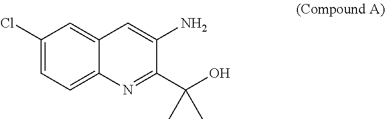 [SMILES: CC(C)(O)c1nc2ccc(Cl)cc2cc1N]

Further illustrating the useful compounds herein are the compounds of formula IIIa:

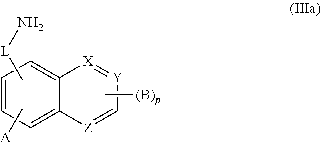

and pharmaceutically acceptable salts thereof, wherein L is a single bond or CH₂; X, Y, and Z are each, independently, N, NH, O, S, CB, CH, or absent, such that one of X, Y, and Z is N or NH; p is 0, 1, 2, or 3;B is a halogen atom, hydroxyl, carbamoyl, aryl or amino;A is

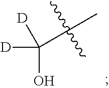 [SMILES: *C([2H])([2H])O]

and D is unbranched lower alkyl; and

Specifically, compounds B and C of paragraph (00046) of WO 2006/127945,and pharmaceutically acceptable salts thereof may be employed in the instant invention.

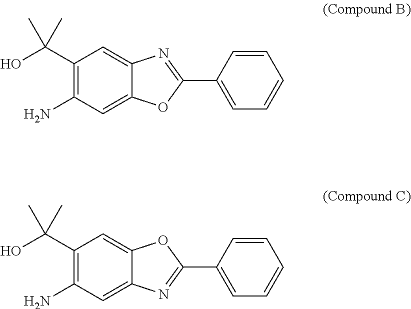 [SMILES: CC(C)(O)c1cc2nc(-c3ccccc3)oc2cc1N ; CC(C)(O)c1cc2oc(-c3ccccc3)nc2cc1N]

The amount of active agent in the composition will vary dependent on the intrinsic activity of the compound. However, for compounds of formula Ia and formula IIIa the amount will generally be from 0.01-1.0% weight/volume and more particularly 0.1% to 0.5% weight/volume. In each case a compound as described above may be used or a pharmaceutically acceptable salt of said compound. The term lipophilic compound is meant to include the compound and its pharmaceutically acceptable salts. A pharmaceutically acceptable salt herein means a salt that is capable of being topically delivered to the eye of a patient. The carrier in the composition is an oligomeric or a polymeric carrier such as a cyclodextrin including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt). Exemplifying an oligomeric or a polymeric carrier is β-cyclodextrin sulfobutylether sodium salt. The amount of β-cyclodextrin sulfobutylether sodium salt in the aqueous preparation may range from about 0.01% to 30% weight/volume. In one illustration the concentration of β-cyclodextrin sulfobutylether sodium salt is 5-25% weight/volume. Further illustrating the concentration of β-cyclodextrin sulfobutylether sodium salt is 9.5-20% weight/volume. In one exemplification the concentration of β-cyclodextrin sulfobutylether is 9.5% weight/volume.

It should be noted that, although the compositions are further illustrated with β-cyclodextrin sulfobutylether, each lipophilic agent may be formulated with each of the excipients. The composition may contain saline and be buffered with for example a phosphate buffer so that the pH of the composition is brought to a pH range of 5.5-8.5 or more particularly a pH of 6.5-7.5. A preservative may optionally be included in the composition. Such preservatives can include both chemical stabilizers and antiseptics. Compounds such as Compounds A are stable under the conditions of use, however other compounds may require the use of other excipients such as anti-oxidants.

A second embodiment of the invention is directed to a method of treating macular degeneration (including dry age-related macular degeneration, GA secondary to dry AMD, wet AMD and Stargardt's disease), by administering a topical formulation of an active lipophilic compound and a carrier to the eye(s) of a patient. In another embodiment, the present invention provides a topical ophthalmic composition for use in treating macular degeneration wherein said composition comprises an active lipophilic compound and a carrier or a pharmaceutically acceptable salt thereof in an aqueous solution. Exemplifying the carrier is β-cyclodextrin sulfobutylether or a pharmaceutically acceptable sat thereof. The active lipophilic compound in each of these embodiments includes those that reduce A2E by reacting with RAL, e.g. the compounds of WO 2006/127945. Examples of such compounds are those described in WO 2006/127945. Exemplifying such compounds are Compounds A, B and C. Lipophilic Compounds which function by other mechanisms such as ACU-4429 are also included. The term lipophilic compound is meant to include both the compound and its pharmaceutically acceptable salts. The excipient in the formulation is oligomeric or polymeric such as a cyclodextrin or a chemically modified cyclodextrin, more particularly including β-cyclodextrin sulfobutylether sodium salt (or potassium salt). It should be noted that, although particularly illustrated with β-cyclodextrin sulfobutylether, the method may be practiced with each lipophilic compound formulated with each excipient. The formulation may be buffered to a pH range of 5.5-8.5 or more particularly a pH of 6.5-7.5. The buffer may be a phosphate buffer. The formulation may be applied as an aqueous solution or a gel dispersion and the solution may be a saline solution. The accumulation of lipofuscin occurs in RPE cells in the back of the eye. Applicants have determined experimentally in animals that the use of topical compositions as described herein deliver pharmacologically active levels of lipophilic active drug safely to the back of the eye after topical ocular administration and are therefore useful in the treatment of macular degeneration (including dry age-related macular degeneration, GA secondary to dry AMD, wet AMD and Stargardt's disease). Efficacious levels are defined as those that reduce A2E accumulation significantly in animal studies.

### Preparation of Compositions of The Invention

Formulations of a lipophilic active agent and an oligomeric or a polymeric carrier are prepared by stirring an aqueous suspension or solution of a polymeric carrier such as a cyclodextrin, or chemically modified cyclodextrin including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium (or potassium) salt with water, saline or phosphate-buffered saline at a temperature from 20° C. to 50° C., but preferably at ambient temperature, for 0.1-24 hr, depending on the polymeric carrier. The lipophilic active agent is added as a solid or neat liquid to the thus formed aqueous suspension or solution of polymeric carrier, and the resulting mixture is stirred at a temperature from 20° C. to 50° C., but preferably at ambient temperature, for 0.1-24 hr, but typically from 0.5-2 hr, depending on the lipophilic active agent. The thus formed formulation of a lipophilic active agent and an oligomeric or a polymeric carrier may then be treated with an aqueous solution of an inorganic or organic acid or an inorganic or organic base to adjust the pH of the solution as desired. To the formulation may also be added preservatives, including both chemical stabilizers and antiseptics.

### Example 1—Preparation of a 0.1% w/v Aqueous Formulation of Compound A with 9.5% w/v β-cyclodextrin Sulfobutylether

The preparation was carried out in a 1000 ml beaker equipped with a stirring bar for mixing. Approximately 60% of the targeted weight of sterile water was added to the beaker. Stirring was adjusted so as not to incorporate air. Anhydrous dibasic sodium phosphate (0.83% of the total batch weight) and sodium phosphate monobasic monohydrate (0.017% of the total batch weight) was added to the vessel and mixed until dissolved. β-cyclodextrin sulfobutylether (9.5% of the total batch weight) was slowly added to the beaker and the solution mixed until dissolved. Compound A (0.1% of the total batch weight) was slowly added and mixed until dissolved. A sample was extracted and the pH measured. If the pH was not in the range 7.3±0.05, adjustment was made with IN aqueous NaOH or IN aqueous HCl. The batch weight was measured and the amount of sterile water needed to bring to final batch weight was determined and added.

### Results of Applying Formulations to the Eyes of Mice and Rabbits.

1) Measurement of levels of ¹⁴C-Compound A in the posterior eyecup of mice.

The target tissue, for the biological activity of compounds which reversibly react with RAL, e.g. Compounds A, B and C, is the outer segment of retinal photoreceptor cells. To demonstrate that topical optical (TO) administration of Compound A delivers therapeutically useful amounts of, for example, Compound A to the retina, C57BL/6 mice, the parent strain of abcr-/-mice (knockout mouse), were treated intraperitoneally (IP) with ¹⁴C-Compound A at 10 mg/kg (“efficacious dose”), specifically a dose that when repeated daily for 56 days reduced A2E formation by 71% (p<0.01) in the abcr-/-mouse. At certain timepoints after topical ocular dosing, the eyes of the mice were enucleated and the posterior eyecup was dissected, extracted and analyzed by liquid scintillation chromatography (LSC) for ¹⁴C-Compound A. Thirty minutes after IP treatment with ¹⁴C-Compound A, the Cₘₐₓ amount of ¹⁴C-Compound A in the posterior eyecup (i.e. the peak concentration at time Tₘₐₓ) was 14.36 μg/g. Fifteen minutes after TO treatment with a single 5 μL eyedrop containing 25 μ(0.5% weight/volume) ¹⁴C-Compound A in a composition containing 20% weight/volume β-cyclodextrin sulfobutylether sodium salt in phosphate-buffered saline, the equivalent amount of ¹⁴C-Compound A in the posterior eyecup was 13.12 μg/g, which is 90% of the Cₘₐₓ value measured in the same ocular tissue after systemic treatment at an efficacious dose.

2) Ocular levels of ¹⁴C-Compound A in the retina and RPE of rabbits.

Dutch belted rabbits were dosed TO with a single 40 μL eyedrop containing either 0.50%, 0.15% or 0.05% weight/volume ¹⁴C-Compound A in 20%, 6% or 2% weight/volume, respectively, β-cyclodextrin sulfobutylether sodium salt in phosphate-buffered saline (Table 1). At different times after dosing the eyes of the rabbits were enucleated, dissected and the amount of ¹⁴C-Compound A in the retina and RPE together was measured by LSC. Each value represents the mean value from 6 eyes. Significant amounts of ¹⁴C-Compound A were found in rabbit retina/RPE within one hour of TO dosing as shown in Table 1.

3) Topical Ocular Dosing with ¹⁴C-Compound A in Cynomolgus Macaques.

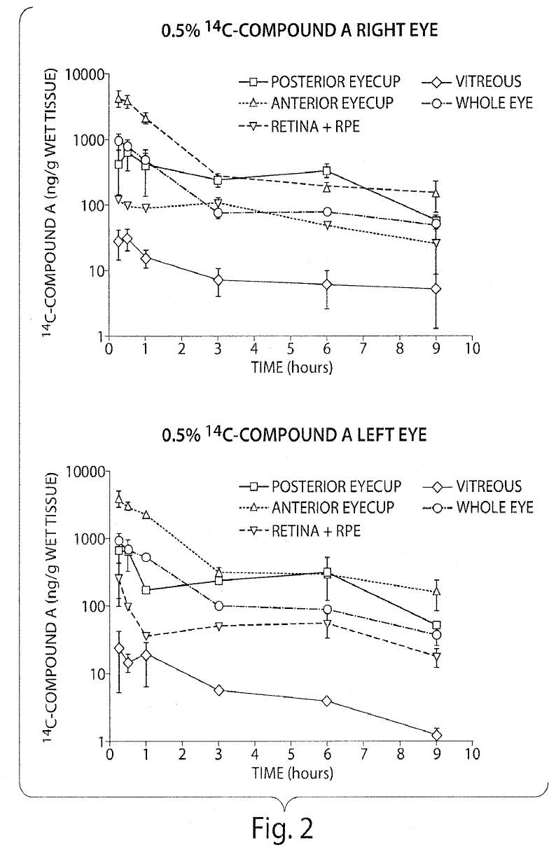

The pharmacokinetics (PK) of Compound A in four intraocular regions of Cynomolgus Macaques was measured after topical dosing of 40 μL per eye of 0.50% weight/volume ¹⁴C-Compound A in β-cyclodextrin sulfobutylether sodium salt in phosphate-buffered saline. Sampling of intraocular regions (vitreous humor, retina+RPE, anterior eyecup, and posterior eyecup) and blood for PK evaluation was conducted at 0.25, 0.5, 1, 3, 6 and 9 hours post dose. FIG. 2 shows the time course of ¹⁴C-Compound A concentration expressed in nanograms (ng) per gram (g) of ocular tissue.

TABLE 1
¹⁴C-Compound A levels in rabbit retina and RPE after acute
TO dosing in β-cyclodextrin sulfobutylether sodium salt.

| | Amount applied | | Amount of ¹⁴C- |
|---|---|---|---|
| Compound A | of ¹⁴C- | | Compound A in |
| concentration in | Compound A | βCSSA¹ | retina and RPE at |
| eyedrop (% w/v) | (ng) | (% w/v) | tₘₐₓ (ng/g) |
| 0.50% | 200 | 20% | 131 |
| 0.15% | 60 | 6% | 47.0 |
| 0.05% | 20 | 2% | 23.1 |

¹β-cyclodextrin sulfobutylether sodium salt;
w/v = weight/volume.

4) Functional biological activity in mice.

The biological activity in the retina of some drugs within the scope of the invention such as Compounds A, B and C cannot be measured non-invasively after topical ocular application because these drugs, e.g. Compounds A, B and C, do not alter the normal function of the retina. For example Compound A does not inhibit the visual cycle nor does it slow the rate of dark adaptation (DA), changes which can be measured by electroretinography (ERG) within minutes after equivalent treatment with a visual cycle inhibitor. The protocol for measuring DA rates by ERG is based on the kinetics of functional visual recovery from photo-bleaching of visual pigment by brief exposure to extremely bright light. Like Compounds A, B and C, retinoids are lipophilic compounds as defined above. As such, retinoids can be used to demonstrate that a small lipophilic compound in a composition containing β-cyclodextrin sulfobutylether sodium salt exhibits biological activity in the retina minutes to hours after topical application to the front of the eye.

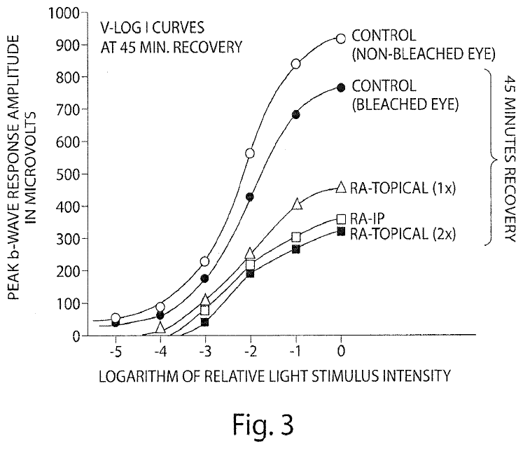

A retinoid useful for this purpose is 13-cis-retinoic acid (RA, also known as isotretinoin). Mice treated IP at 20 mg/kg with RA show visual cycle inhibition (FIG. 3). Independent studies demonstrate that the visual cycle inhibition decreases A2E synthesis (Radu R A, Mata N L, Nusinowitz S, Liu X, Sieving P A, Travis G H. Treatment with isotretinoin inhibits lipofuscin accumulation in a mouse model of recessive Stargardt's macular degeneration. Proc Natl Acad Sci 2003;100:4742). However, mice treated TO with one or two 30 μL eyedrops of a composition containing 0.2% weight/volume RA and 20% weight/volume β-cyclodextrin sulfobutylether sodium salt in phosphate-buffered saline showed a dose-responsive inhibition of DA similar to that observed after IP dosing (FIG. 3).

What is claimed is:

1-6. (canceled)

7. A method for treating macular degeneration comprising administering a topical ophthalmic composition to the eye(s) of a patient, wherein said topical composition comprises a lipophilic compound selected from the group consisting of Compound A, Compound B and Compound C and a carrier selected from β-cyclodextrin sulfobutylether or a pharmaceutically acceptable salt thereof in an aqueous solution.

8. The method of claim 7 wherein the lipophilic compound is Compound A or a pharmaceutically acceptable salt thereof.

9. The method of claim 8 wherein the carrier is β-cyclodextrin sulfobutylether sodium salt.

10. The method of claim 9 wherein the solution is an aqueous saline solution phosphate buffered to a pH of 6.5-7.5.

11. The method of claim 10 wherein the concentration of β-cyclodextrin sulfobutylether sodium salt is 9.5-20% w/v.

12. The method of claim 11 wherein the concentration of β-cyclodextrin sulfobutylether is 9.5% w/v.

13. The method of claim 12 wherein the form of macular degeneration being treated is dry AMD.

14. The method of claim 12 wherein the form of macular degeneration being treated is geographic atrophy secondary to dry AMD.

15. The method of claim 12 wherein the form of macular degeneration being treated is wet AMD.

16. The method of claim 12 wherein the form of macular degeneration being treated is Stargardt's disease.
